(19) United States
(12) Patent Application Publication
(10) Pub. No.: US 2007/0207535 A1
(43) Pub. Date: Sep. 6, 2007

(54) Reaction Container, and Reaction Device and Detection Device Using the Reaction Container

(75) Inventor: Yuichiro Matsuo, Hachioji-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATI0N, Tokyo (JP)

(21) Appl. No.: 11/629,736

(22) PCT Filed: Jun. 14, 2005
(86) PCT No.: PCT/JP05/11210

§ 371 Date: Dec. 15, 2006

(30) Foreign Application Priority Data
Jun. 15, 2004 (JP) 2004-176779
Sep. 1, 2004 (JP) 2004-254417

(51) Int. Cl. C12M 3/00

(52) U.S. Cl. 435/287.1

(57) ABSTRACT

A reaction container 101 having a substrate (102) in which a probe to detect a biologically associate substance is phase-locked, the reaction container (101) comprising a correcting section (105, 106, 107, 108, 109) to align a detection direction of a detector (129) to detect a reaction of the substrate and a direction of the substrate.

### TECHNICAL FIELD

The present invention relates to a reaction container provided to detect a biologically associated substance and a reaction device and a detection device using the reaction container.

### BACKGROUND ART

In recent years, gene analysis for many kinds of lives including a human being or a plenty of plants such as a rice crop has been underway. Lately, there has been developed an inspection method using a DNA chip or a DNA micro-array on which DNAs are regularly arrayed at a semiconductor or the like. In this inspection method, a plurality of genes can be inspected at the same time.

WO 03/005013 discloses a micro-array chip having a structure in which a reaction solution is repeatedly passed through a reaction section by a syringe or a piston by using a porous substrate.

The micro-array chip disclosed in WO 03/005013 comprises a construction in which: the reaction solution is dispensed in a reaction container composed of multiple chips from above a substrate of the micro-array chip; a reaction state is observed at an upper part of the micro-array chip; and pressurization and de-pressurization are carried out by a syringe pump at a lower part of the chip. In the micro-array chip having such a construction, in general, an illumination light (excited light) is emitted to a phase-locked probe (hereinafter, referred to as a “spot”) after hybridization has finished in the reaction container; and a fluorescence quantity generated from the spot is acquired as an image by using a cooling CCD camera or the like. Then, a fluorescence luminance is measured by using analysis software or the like, and it is determined which spot and how many spots has (have) been developed based on known spot information (position or base array).

However, in the case where a fluorescence image after hybridized is acquired, matching between spot information and the spot luminance of the acquired fluorescence image cannot be obtained by the analysis software or the like if a relationship between an image pickup direction (vertical and horizontal directions) of, for example, a cooling CCD camera, and a spot array direction is not clarified, so that it is impossible to determine which spot is being analyzed. In the microchip array described in WO 03/005013, spotted multiple chips are provided inside of a chip holder mounted on a syringe piston, and the reaction solution is vertically reciprocated while the solution passes through the inside of the multiple chips by means of syringe movement. After a sample has been hybridized by this movement, the luminance of the multiple chip spot located inside of the chip holder is measured. In this case, there is not proposed means for specifying a positional relationship between the syringe piston and the chip holder. Thus, there occurs a case in which the photography direction does not match the multiple chip spot array direction when fluorescence photography has been carried out by the cooling CCD camera or the like. Therefore, in the case where a fluorescence image has been photographed, the spot array direction relevant to an image pickup face is not identified. Thus, there is a possibility that a correlation between the spot information and an actual spot luminance cannot be obtained and fluorescence luminescence analysis cannot be carried out.

### DISCLOSURE OF INVENTION

It is an object of the present invention to provide a technique for matching a direction of a pickup image of detection means such as a CCD camera and a spot array direction, and specifically, a technique capable of sensing or correcting a direction of a reaction container.

A reaction container having a substrate in which a probe to detect a biologically associate substance is phase-locked, according to a first aspect of the invention is characterized by comprising a correcting section to align a detection direction of a detector to detect a reaction of the substrate and a direction of the substrate.

A detection device according to a second aspect of the invention is characterized by comprising:

a reaction container having a substrate in which a probe to detect a biologically associated substance is phase-locked; a detector which detects a reaction of the substrate; and a correcting section to match a direction of the substrate and a detection direction of the detector.

A reaction device according to a third aspect of the invention is characterized by comprising:

a reaction container having a substrate in which a probe to detect a biologically associated substance is phase-locked; a pressure transmitting section which transmits to the reaction container a pressure for charging and discharging a sample solution; a temperature adjusting section which adjusts a temperature of the sample solution contained in the reaction container; and a correcting section which causes the reaction container to be oriented in a predetermined direction.

### BRIEF DESCRIPTION OF DRAWINGS

FIG. 1 is a diagram schematically depicting a reaction container according to a first embodiment of the present invention, and an inspection device having the reaction container mounted thereon.

FIG. 2 is an enlarged diagram showing a portion which includes an engagement section and the reaction container.

FIG. 3 is an enlarged view of a coupling section and a reflection sensor.

FIG. 4 is a diagram showing a schematic control circuit according to the first embodiment.

FIG. 5 is a top view of the reaction container.

FIG. 6 is a diagram schematically depicting a reaction container and an inspection device according to a second embodiment of the present invention.

FIG. 7 is a diagram showing essential portions of FIG. 6 in details.

FIG. 8 is a diagram showing a schematic control circuit according to the second embodiment.

FIG. 9 is a detailed view showing a coupling section according to the second embodiment.

FIG. 10A is a view showing an output voltage of a Hall element depending on a position of a micro-magnet.

FIG. 10B is a view schematically depicting a position of the micro-magnet in the Hall element in the case where the result shown in FIG. 10A has been obtained.

FIG. 11A is a view showing an output voltage of a Hall element depending on a position of a micro-magnet.

FIG. 11B is a view schematically depicting a position of the micro-magnet in the Hall element in the case where the result shown in FIG. 11A has been obtained.

FIG. 12 is a diagram schematically depicting a reaction device and an inspection device according to a third embodiment of the present invention.

FIG. 13 is a detailed view showing a coupling section and an engagement section according to the third embodiment.

FIG. 14 is a diagram showing a schematic control circuit according to the third embodiment.

FIG. 15 is a view showing a modification of the reaction container.

FIG. 16 is a view showing another modification of the reaction container.

FIG. 17 is a view showing still another modification the reaction container.

### BEST MODE OF CARRYING OUT THE INVENTION

Hereinafter, embodiments of the present invention will be described with reference to the accompanying drawings.

### FIRST EMBODIMENT

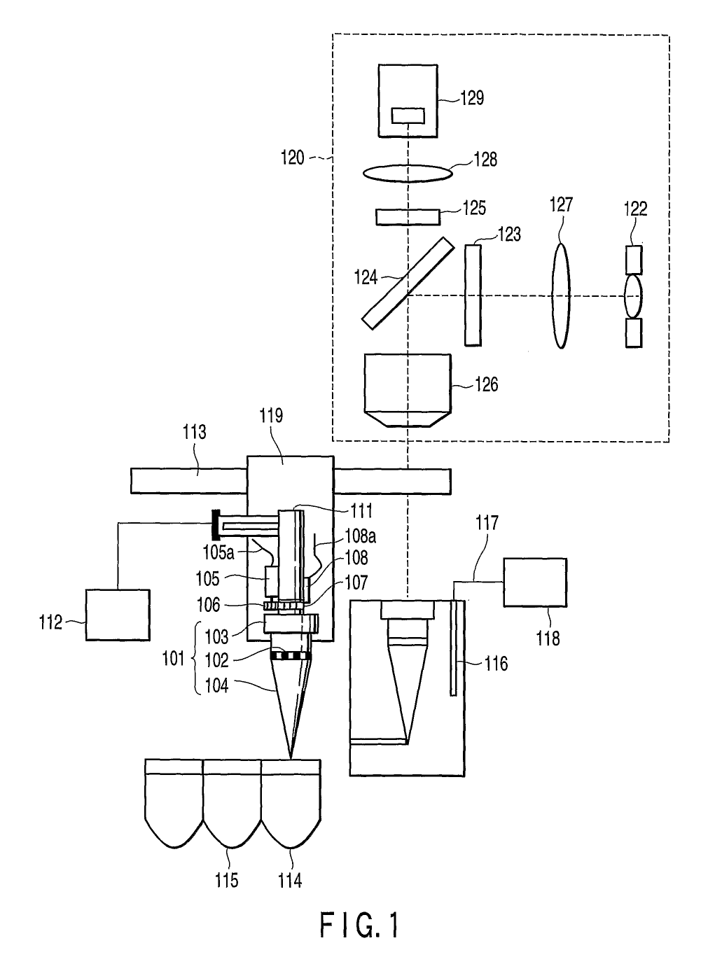

A first embodiment of the invention will be described with reference to FIGS. 1 to 5.

FIG. 1 is a diagram schematically depicting a reaction container according to the first embodiment of the invention, and an inspection device having the reaction container mounted thereon. In FIG. 1, a reaction container 101 comprises: a three-dimensional substrate 102 in which a probe for detecting a biologically associated substance is phase-locked; a coupling section 103 to be coupled with the inspection device; and a solution charge section 104 which charges a solution. In the present specification, the “three-dimensional substrate” used herein denotes a substrate for phase-locking a probe. Any material or structure can be used as a three-dimensional substrate as long as it widens in a three-dimensional direction and has a structure capable of substantially passing a liquid. For example, a variety of filters and porous bodies can be used as a three-dimensional substrate. In particular, it is preferable to use a porous body whose Si wafer has been etched, a hollow thread, a metal oxide film, a bead, or a capillary. In addition, in the three-dimensional substrate 102 a plurality of reaction regions are formed in which probe molecules which supplement a target substrate are phase-locked in size of, for example, 120 μm.

The three-dimensional substrate 102 is allocated between the coupling section 103 and the solution charge section 104. In the present embodiment, the coupling section 103 and the solution charge section 104 are coupled with each other while the three-dimensional substrate 102 is sandwiched therebetween. The coupling section 103 and the solution charge section 104 are made of a non-transparent material.

The inspection device comprises: an engagement section 111 which engages with the coupling section 103 of the reaction container 101; a pressure transmitting section 112 which transmits a pressure to the reaction container 101 through the engagement section 111; and a two-shaft robot which movably supports the engagement section 111. The inspection device further comprises: a sample container 114 which houses a sample solution; and a cleaning solution container 115 which houses a cleaning solution. The sample container 114 is composed of, for example, a micro tighter plate. The cleaning solution container 115 is composed of, for example, a micro tighter plate and a bottle. The coupling section 103 of the reaction container 101 has an opening end. When the engagement section 111 engages with the opening end of the coupling section 103, the reaction container 101 is mounted on the engagement section 111. By means of an electrically driven slider 113, the reaction container 101 mounted on the engagement section 111 can be moved in a horizontal direction. Further, by means of an electrically driven slider 119, the reaction container 101 mounted on the engagement section 111 can be moved in a vertical (up and down) direction.

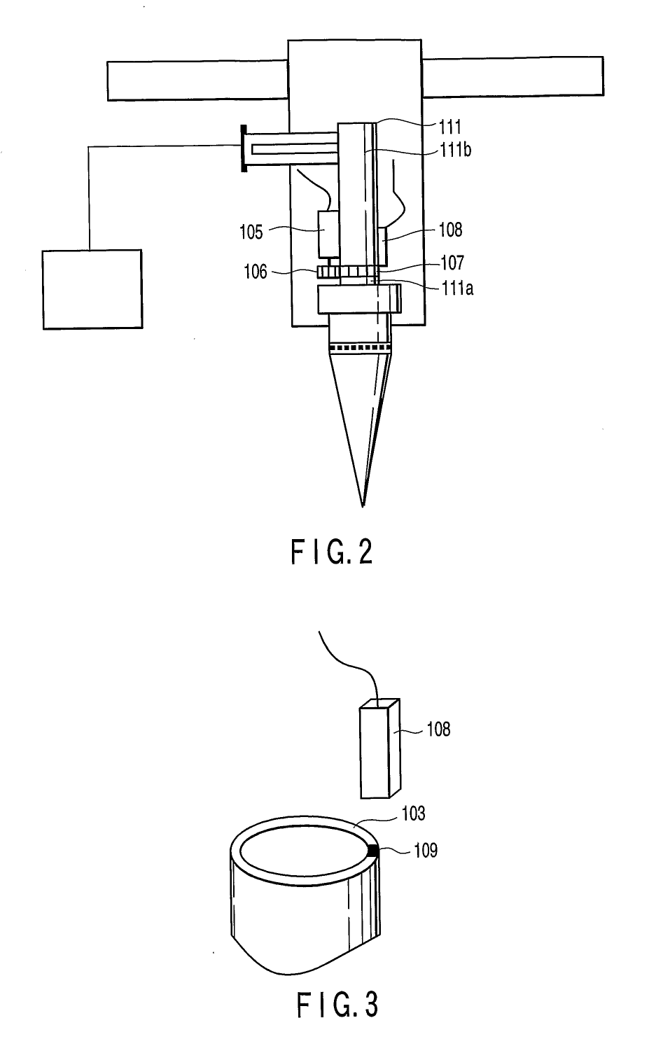

The pressure transmitting section 112 coupled with the reaction container 101 mounted on the engagement section 111 transmits a pressure to the reaction container 101 via the engagement section 111. In this manner, the sample solution contained in the sample container 114 and the cleaning solution contained in the cleaning solution container 115 can be charged in the reaction container 101 or the sample solution or cleaning solution contained in the reaction container 101 can be discharged, via the solution charge section 104. Referring to FIG. 2, the engagement section 111 will be further described in detail. FIG. 2 is an enlarged diagram showing a portion which includes the engagement section 111 and the reaction container 101. The engagement section 111 comprises an engagement section upper part 111b and a rotatable engagement section lower part 111a. A gear 117 is mounted at the engagement section lower part 111a. Further, the engagement section upper part 111b has a reflection type optical sensor 108 and a small sized motor 105, and a pinion gear 106 mating with the gear 107 is mounted at a distal end of the small sized motor. The pinion gear 106 rotates, whereby the gear 107 rotates and the engagement section lower part 111a rotates.

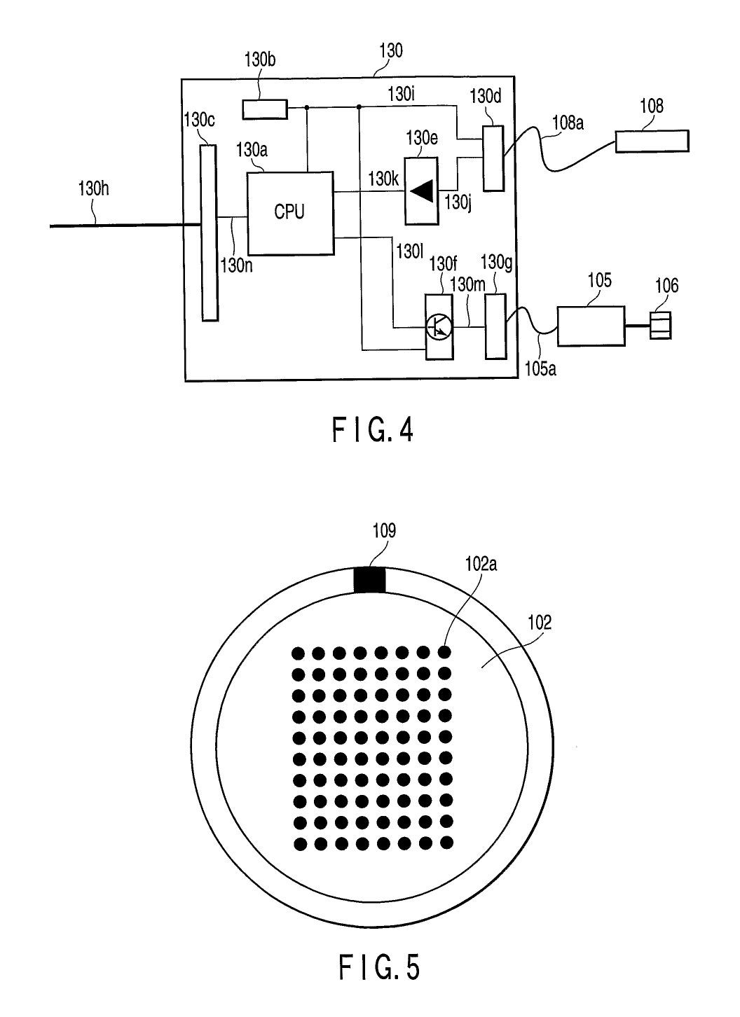

The coupling section 103 comprises a reflection member 109 at an end face thereof, as shown in FIG. 3, and the direction of the reaction container 101 is sensed by the reflection member 109. For example, when the reflection member 109 is located immediately beneath the reflection type optical sensor 108, the reflection type optical sensor 108 outputs an H-level signal. In this manner, the direction of the reaction container 101 is sensed to be a predetermined direction. The small sized motor 105 and the reflection type optical sensor 108 are connected to a computer (not shown) via a control board 130 shown in FIG. 4. As shown in FIG. 4, the control board 130 has a CPU 130a, a power supply module 130b, a communication connector 130c, a sensor IF connector 130d, a buffer IC 130e, a motor driver IC 130f, a motor IF connector 130g, and an RS-232C cable 130h. The CPU 130a carries out control and signal processing of each section. The power supply module 130b supplies power to each part mounted on the control board 130. An operation of each section will be described later.

The three-dimensional substrate 102 allocated between the coupling section 103 and the solution charge section 104 is managed such that the three-dimensional substrate 102 is mounted in a predetermined direction relevant to the reflection member 109 at the time of its assembling, as shown in FIG. 5. Namely, the substrate and charge section are managed in its assembling process such that a position of a first spot 102a is always set to the same location relevant to the reflection member 109. The reaction container 101 is generally provided in the form such that, for example, 96 reaction containers are inserted into an opening of a tray (not shown). The pressure transmitting section 112 transmits a pressure to the reaction container 101 mounted on the engagement section 111, whereby the sample solution contained in the sample container 114 and the cleaning solution contained in the cleaning solution container 115 are charged in the reaction container 101 or the sample solution and cleaning solution contained in the reaction container 101 is discharged, via the solution charge section 104.

The inspection device further comprises a heater 116, a temperature measuring resistor 117, and a temperature control section 118. The heater 116 controls a sample temperature during a hybridization reaction or a cleaning solution temperature during cleaning. The temperature measuring resistor 117 measures a temperature of the heater 116. The temperature control section 118 controls the temperature of the heater 116 on the basis of information obtained by the temperature measuring resistor 117. The heater 116 has a space for housing the reaction container 101. The reaction container 101 mounted on the engagement section 111 is moved into the space formed at the heater 116 by the electrically driven sliders 113 and 119 as required. The heater 116, the temperature measuring resistor 117, and the temperature control section 118 configure temperature adjusting means for adjusting a temperature of the solution contained in the reaction container 101. The temperature adjusting means, the engagement section 111, and the pressure transmitting section 112 configure a reaction device which accelerates a hybridization reaction.

The inspection device further comprises an observation optical system 120 for optically observing the three-dimensional substrate 102 contained in the reaction container 101. The observation optical system 120 is, for example, a fluorescence observation optical system. The observation optical system 120 comprises a light source 122, an excitation filter 123, a dichroic mirror 124, and a fluorescence filter 125. The light source 122 emits a light having a visual wavelength. The excitation filter 123 selectively transmits a wavelength for exciting a fluorescence substance coupled with a sample molecule from the light generated from the light source. The dichroic mirror 124 reflects the light transmitted through the excitation filter 123 and selectively transmits a fluorescence generated from the fluorescence substance. The fluorescence filter 125 selectively transmits the fluorescence generated from the fluorescence substrate and transmitted through the dichroic mirror 124. The observation optical system 120 further comprises an objective lens 126, an illumination optical system 127, an image focusing optical system 128, and a CCD 129. The objective lens 126 is provided for optically detecting trapping of a sample molecule for a probe molecule placed on the three-dimensional substrate 102. The illumination optical system 127 guides the light from the light source 122 to the three-dimensional substrate 102 via the excitation filter 123 and the dichroic mirror 124. The image focusing optical system 128 is provided for focusing as an image the light from the three-dimensional substrate 102, the light being captured by the objective lens 126. The CCD 129 converts the optical image focused by the image focusing optical 128 into an electrical signal. The CCD camera 129 has a function for dividing and optically measuring the inside of an image pickup area in a predetermined number. The fluorescence from each spot can be optically measured independently by using the dividing and optically measuring function.

Now, an operation of the inspection device according to the embodiment will be described in accordance with a general inspection process.

A nucleus is extracted from a living sample, the extracted nucleus is labeled by a fluorescence substance such as FITC, and then, the labeled nucleus is dissolved in a buffer solution (hereinafter, this solution is referred to as “sample solution”). After the dissolved sample solution is dispensed into the sample container 114, the sample container 114 is placed at a sample solution setting position. A cleaning solution is dispensed into the cleaning solution container 115, and the cleaning solution container 115 is placed at a cleaning solution container setting position. The reaction container 101 is set at a predetermined position (for example, on a tray). Next, in order to mount on the engagement section 111 the reaction container 101 set at the predetermined position (not shown), the electrically driven sliders 113 and 119 are operated.

When the engagement section 103 of the reaction container 101 engages with the engagement section 111, and the reaction container 101 is mounted on the engagement section 111, a computer (not shown) starts an operation of correcting a mount position (rotary angle) of the engagement section and the reaction container 101. The computer (not shown) transmits a position correcting command via the RS232C cable 130h. The CPU 130a having received a command reads a signal from the reflection type optical sensor 108 via a signal line 108a, the sensor IF connector 130d, a signal line 130j, the buffer IC 130e, and a signal line 130k. At this time, if a signal level does not reach an H level, the CPU 130a rotates the motor 105 via a motor drive signal 130l, the motor driver IC 130f, the motor IF connector 130g, and a drive cable 105a. By this operation, the reaction container 101 rotates together with the engagement section lower part 111a. While in this rotating operation, the CPU 130a continues checking an output signal of the reflection type optical sensor 108. After the reflection member 109 has reached immediately beneath the reflection type optical sensor 108, the CPU stops the motor 105 when an output of the reflection type optical sensor becomes H.

Subsequently, the inside of the reaction container 101 is de-pressurized by means of the pressure transmitting section 112 via the engagement section 111, and the sample solution is sucked from the sample container 114. In the solution charge section 104 of the reaction container, it is preferable that a portion lower than a phase-locked carrier is formed in a tapered shape, as shown in FIG. 1 or FIG. 2. This is because the residual quantity of the solution is reduced, in particular, when the sample solution is sucked from a micro tighter plate or the like. In addition, when an air layer is sucked in after suction of the sample solution, no sample solution drips from the reaction container during movement to the temperature control section. Further, also when the sample solution is moved vertically of the three-dimensional substrate, such a tapered shape is preferable because no sample solution drips from the reaction container.

Now, the electrically driven sliders 113 and 119 are operated in order to mount the reaction container 101 so as to come into intimate contact with a recess of the heater 116. Subsequently, the heater 116 is controlled to a desired temperature in order to carry out hybridization. Then, pressurization and de-pressurization inside of the reaction container 101 by use of the pressure transmitting section 112 are repeatedly carried out, the sample solution is moved vertically of the three-dimensional substrate 102, and the nucleus contained in the sample solution is hybridized. After hybridization reaction has finished, the inside of the reaction container 101 is pressurized via the engagement section 111 by means of the pressure transmitting section 112 and the sample solution is discarded into the sample container 114. Next, the inside of the reaction container 101 is de-pressurized via the engagement section 111 by means of the pressure transmitting section 112, and the cleaning solution is sucked from the cleaning solution container 115. The pressurization and de-pressurization inside of the reaction container 101 by use of the pressure transmitting section 112 are repeatedly carried out, whereby the three-dimensional substrate 102 is cleaned with the cleaning solution, and the nucleus which has not been coupled with a probe molecule, the nucleus being contained in the sample solution, is washed out.

After a hybridization has finished, the electrically driven sliders 113 and 119 are operated, the reaction container 101 is removed from the engagement section 111, and the three-dimensional substrate 102 is exposed. In this manner, a reaction result can be read, the observation optical system 120 allocated above the reaction container 101 is controlled to emit an excited light to the three-dimensional substrate 102, the light (for example, fluorescence) from the probe spot on the three-dimensional substrate 102 is photographed as electronic image data by the CCD camera 129, and data on an image or light quantity on the probe spot is stored. Then, the thus stored data is analyzed, and the developing state, variance, polymorphism and the like of the nucleus contained in the sample solution are inspected.

In the first embodiment, the reflection member 109 is provided so that the reaction container 101 is always oriented in a fixed direction. Thus, the direction of the reaction container 101 is always stabilized relevant to the image pickup face of the CCD camera 129, thereby making it possible to carry out precise data analysis based on a positional relationship of the photographed spot.

### SECOND EMBODIMENT

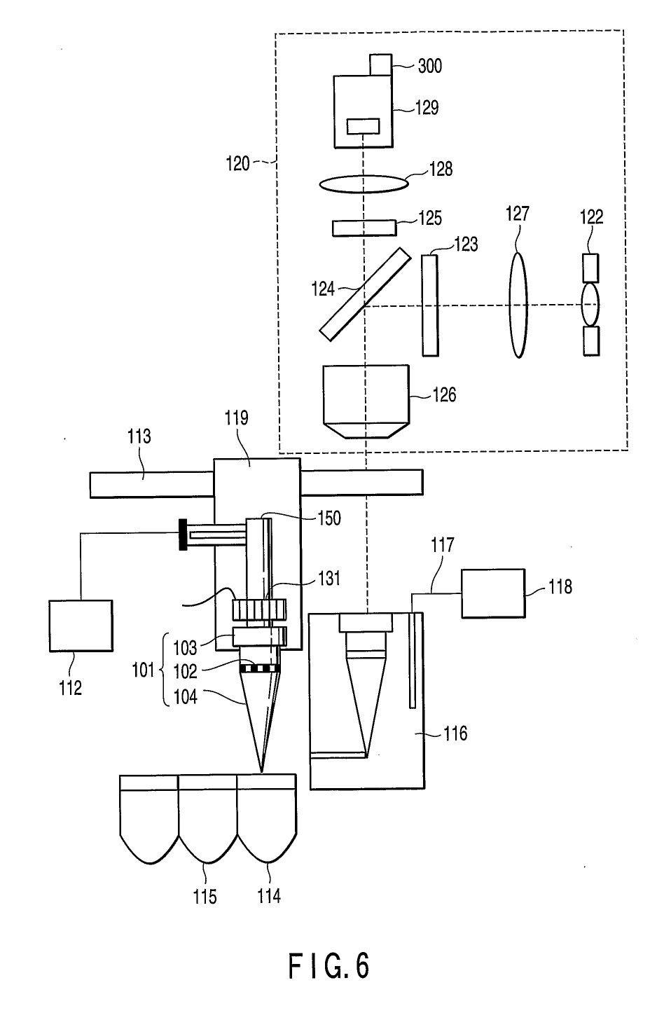

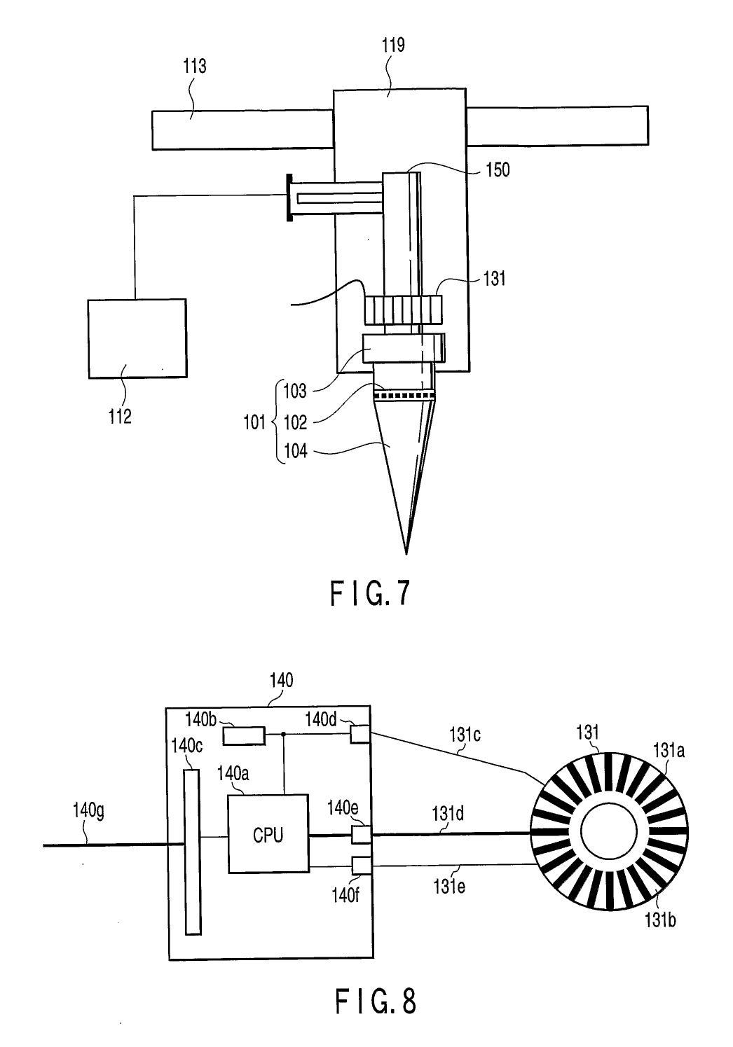

A second embodiment of the invention will be described with reference to FIGS. 6 to 11B. FIG. 6 is a diagram schematically depicting a reaction container and an inspection device according to the second embodiment of the invention. FIG. 7 is a detailed diagram showing essential portions of FIG. 6. In FIGS. 6 to 11B, like constituent elements shown in FIGS. 1 to 5 are designated by like reference numerals, and a description and an explanation of the constituent elements relating to the same operation are omitted here.

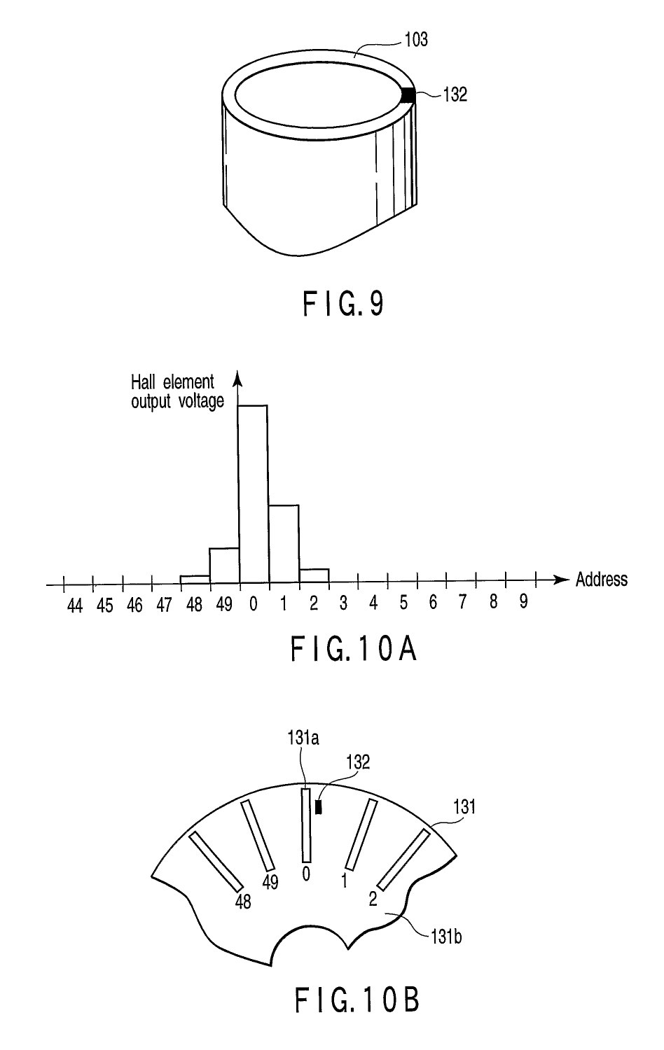

In this embodiment, as shown in FIGS. 6 and 8, a position sensor 131 is mounted on an engagement section 150. The position sensor 131 has concentrically packaged thereon 50 Hall elements (micro sensors) 131a packaged on a ring-shaped printed circuit board 131b. In addition, a micro magnet 132 is embedded in the coupling section 103 as shown in FIG. 9. FIG. 8 is a diagram showing a mechanism for detecting a position (rotary angle) of the reaction container when the engagement section 150 and the coupling section 103 in the embodiment have been engaged with each other. As shown in FIG. 8, the position sensor 131 is formed in a circular shape, and the 50 Hall elements 131a are concentrically arranged (at intervals of 7.2 degrees) on the printed circuit board 131b having a circular ring shape. A control board 140 comprises a CPU 140a, a power supply module 140b, a communication connector 140c, a power supply connector 140d, a sensing connector 140e, a position detecting signal connector 140f, and an RS-232C cable 140g. In FIG. 8, the position sensor 131 is viewed from the side of the reaction container 101 in the case where the sensor has been mounted on the engagement section 150.

A description will be given with respect to an operation of the inspection device according to the second embodiment configured as described above.

When the engagement section 150 and the coupling section 103 engage with each other, a computer (not shown) sends a command for sensing an output from which Hall element 131 is the largest to the CPU 140a of the control board 140 via the RS232C cable 140g. The CPU 140a having received the command uses, for example, a 6-bit signal line of the signal line 131d to monitor an output voltage of the signal line 131e and store the output voltage in a memory of the CPU 140 while incrementing an address allocated to each Hall element 131a. Each Hall element 131a to which power is supplied from a power supply line 131c has a characteristic of changing an output voltage in response to the intensity of a magnetic field received from the micro magnet 132. As the intensity of the magnetic field received from the micro magnet 132 increases, the output voltage from the Hall element 131a increases.

When the output voltage of the signal line 131e has been monitored, the CPU 140a calculates a shift quantity of an angle of the micro magnet from an address of each of the Hall elements 131a whose output voltage has been the highest and the second highest and a ratio of both of the output voltages. In this case, the Hall elements 131 whose output voltages have been the highest and the second highest are adjacent to each other. The image pickup direction of the CCD camera 129 and the address of the Hall element 103a are associated with each other in relationship. When the micro magnet reaches address 0, the direction (rotary angle) of the reaction container and the image pickup direction of the CCD camera coincide with each other. In the present embodiment, permissible addresses range from 0 to 49. A specific example thereof will be shown below.

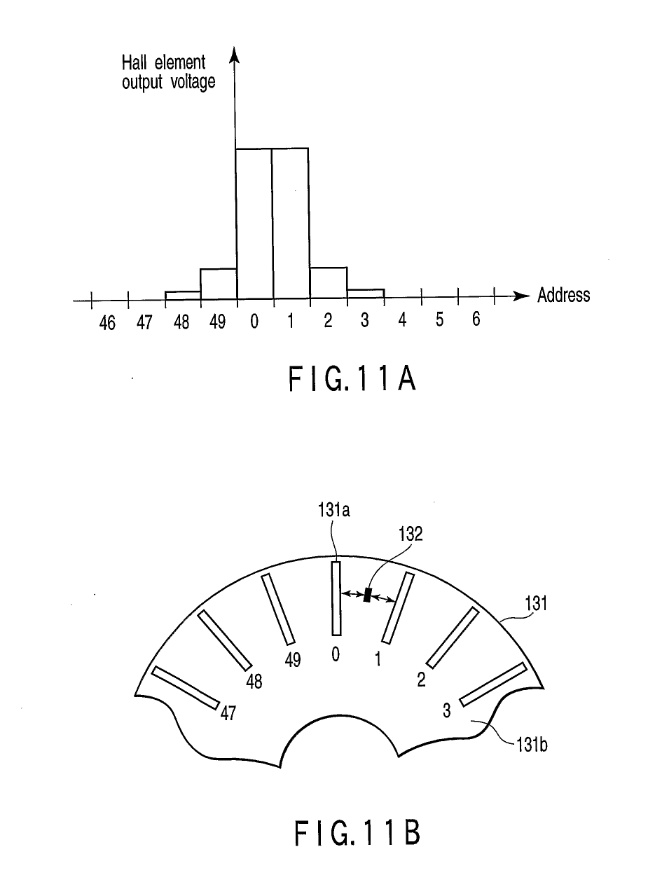

For example, assume that the address of the Hall element 131a indicating the largest voltage value has been set to 1 and that the address of the Hall element 131a indicating the second largest voltage value has been set to 2. From this result, it is found that the position of the micro magnet 132 ranges from 7.2 degrees to 14.4 degrees in terms of an angle from address 0. Further, a precise position of the micro magnet 132 is obtained by computation using a ratio between a voltage of the Hall element 131a having address 1 and a voltage of the Hall element 131a having address 2. In this case, it is possible to obtain an angle as a position of the micro magnet 132 by means of computation in order of 1/10 of an allocation gap (7.2 degrees) of the Hall element 131a, for example. FIGS. 10A and 11A each are a graph in the case where the output voltage of the Hall element 131a is taken along a vertical axis, and the address of the Hall element 131a is taken along a horizontal axis. In FIG. 10A, the output voltage of the Hall element 131a having address 0 is the largest and the output voltage of the Hall element 131a having address 1 is the second largest. In FIG. 11A, the output voltages of the Hall elements 131a having address 0 and address 1 are equal to each other. FIGS. 10B and 11B are views each schematically depicting a position set in the Hall element 131a of the micro magnet 132 in the case where the results shown in FIGS. 10A and 11A have been obtained. As shown in FIGS. 10B and 11B, respectively, it is found that the micro magnet 132 is positioned in the vicinity of the Hall element 131a having address 0 in the case of FIG. 10B, and that the micro magnet 132 is positioned at an intermediate position between the Hall elements 131a having address 0 and address 1 in the case of FIG. 11B.

The CPU 140 calculates a shift between the image pickup direction of the CCD camera 129 and the position (rotary angle) of the reaction container 101 actually mounted on the engagement section 150 on the basis of the position of the micro magnet 132 obtained by the above described method, and transmits angle information to the computer (not shown) via the RS232C cable 140g. After a hybridization has finished, the electrically driven sliders 113 and 119 are operated, and the reaction container 101 is removed from the engagement section 150, whereby the three-dimensional substrate 102 is exposed, in the same manner as in the first embodiment. Next, the CCD camera 129 is rotated by a CCD camera rotation mechanism 300 by a shift (rotary angle) calculated by the CPU 140a. In this manner, the direction of the CCD camera 129 coincides with that of the reaction container 101. Then, as in the first embodiment, the observation optical system 120 located above the reaction container 101 is controlled to emit an excited light, and the light from the probe spot on the three-dimensional substrate 102 is photographed as electronic image data by the CCD camera 129. Thereafter, data on an image or light quantity data on the probe spot is stored. The thus stored data is analyzed, and the developing state, variance, polymorphism and the like of the nucleus included in the sample solution are inspected.

In the second embodiment, the direction of the reaction container 101 is sensed, so that the direction of the CCD camera 129 and the direction of the reaction container 101 coincide with each other. That is, when the engagement section 150 and the coupling section 103 engage with each other, the shift of the rotary angle relevant to the image pickup direction is corrected during photography even in the case where the direction of the reaction container 101 does not coincide with the image pickup direction of the CCD camera 129. This makes it possible to carry out precise data analysis from a spot positional relationship.

### THIRD EMBODIMENT

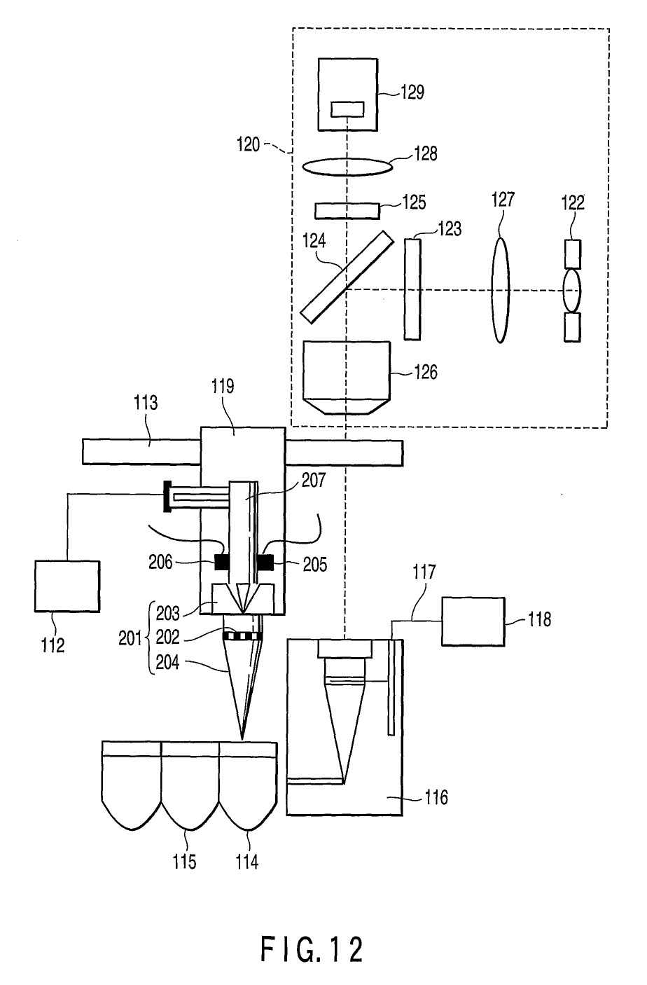

A third embodiment of the invention will be described with reference to FIGS. 12 to 14.

FIG. 12 is a diagram schematically depicting a reaction container and an inspection device according to the third embodiment of the invention. In the third embodiment, like constituent elements shown in the first and second embodiments are designated by like reference numerals, and a description and an explanation of the constituent elements relating to the same operation are omitted here.

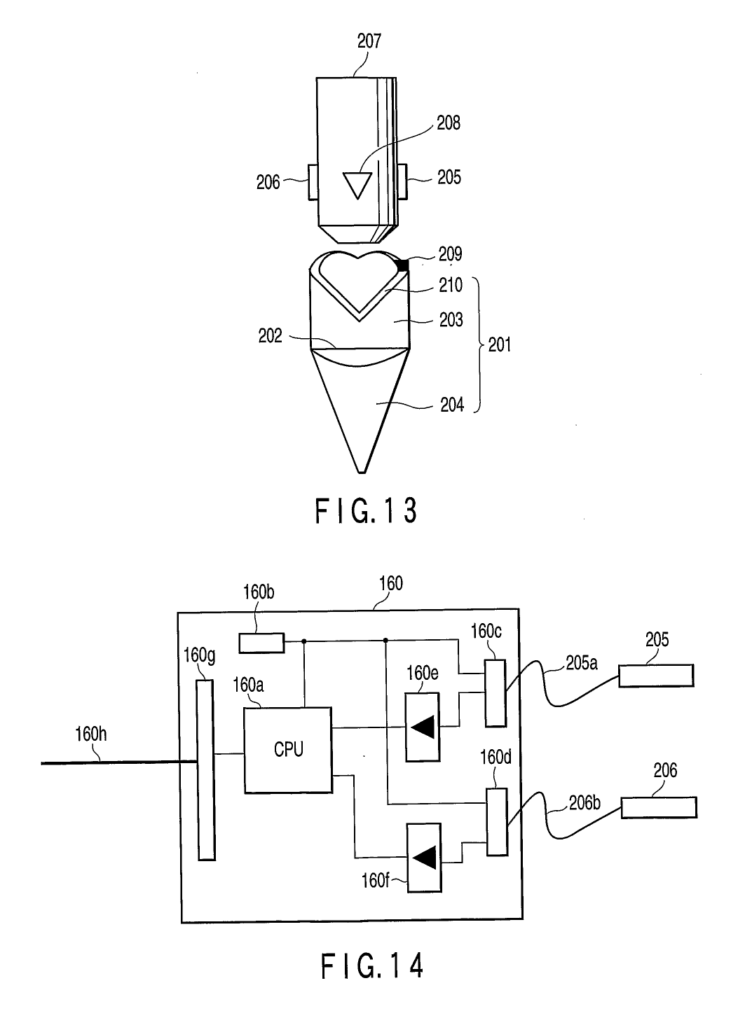

In FIGS. 12 and 13, an engagement section 207 comprises reflection type optical sensors 205, 206 and an engagement guide section 208. A guide cutout portion 210 is provided in a horizontally symmetrical manner at a coupling section 203 of a reaction container 201. The engagement guide section 208 carries out position correction during engagement along the guide cutout section 210. Further, the coupling section 203 comprises a reflection member 209 composed of the reflection type optical sensors 205, 206. The reflection type optical sensors 205, 206 are connected to a control board 160 as shown in FIG. 14. As shown in FIG. 14, the control board 160 comprises a CPU 160a, a power supply module 160b, a sensor IF connector 160c, a sensor IF connector 160d, a buffer IC 160e, a buffer IC 160f, a communication connector 160g, and an RS-232C cable 160h.

A description will be given with respect to an operation of the inspection device according to the third embodiment configured as described above.

When the engagement section 207 engages with the coupling section 203 of the reaction container 201, the engagement section 208 engages with the reaction container 201 while rotating the container by pressing the guide cutout section 210 of the coupling section 203. At a time point at which engagement has terminated, the engagement guide section 208 always abuts against the deepest portion of any guide cutout section 210 by means of the guide cutout section 210 of the horizontally symmetrical coupling sections 203. In this state, the reflection member 209 is designed so as to be located immediately beneath either of the reflection type optical sensors 205 and 206. The three-dimensional substrate 202 allocated between the coupling section 203 and the solution charge section 204 is managed such that the three-dimensional substrate 202 is mounted in a predetermined direction relevant to the reflection member 209 at the time of assembling the substrate. As a result, at a time point at which engagement has terminated, the direction of the three-dimensional substrate 202, namely, the spot array direction is mounted in a direction of 0 degree or 180 degrees relevant to the image pickup direction of the CCD camera 129.

When engagement terminates, a computer (not shown) inquires the CPU 160a as to which reflection type optical sensor output is set to H via the RS-232C cable 160h. The CPU 160a checks outputs of the buffer ICs 160e, 160f having received output signals via the reflection type optical sensors 205, 206 and signal lines 205a, 206a. In the present embodiment, the output from the reflection type optical sensor 205 is set to H and is set in a state in which the output is rotated by 180 degrees with respect to the image pickup direction of the CCD camera 129. The CPU 160a transmits the information as correction information to the computer (not shown) via the RS-232-C cable 160h. After a hybridization work has completed, the electrically driven sliders 113 and 119 are operated, and the reaction container 201 is removed from the engagement section 207, whereby the three-dimensional substrate 202 is exposed, in the same manner as in the first embodiment. In this manner, as is in the first embodiment, the observation optical system 120 located above the reaction container 201 is controlled to emit an excited light, and the light from the probe spot on the three-dimensional substrate 202 is photographed as electronic image data by the CCD camera 129. After an image has been rotated by image processing based on the correction information transmitted from the CPU 160a, data on the image or light quantity on the probe spot is stored in the present embodiment.

Then, the thus stored data is analyzed, and the developing state, variance, polymorphism and the like of the nucleus contained in the sample solution are inspected. With such a configuration, when the engagement section 207 and the coupling section 203 engage with each other, a deviation of the rotary angle of the photographed image can be corrected even if the direction of the reaction container 201 does not match the image pickup direction of the CCD camera 129. Therefore, precise data analysis can be carried out from a spot positional relationship.

The present invention is not limited to the above-described embodiments. At the stage of carrying out the invention, various modifications can occur without departing from the spirit of the invention.

For example, the reflection type optical sensor may be changed to a Hall element, and the reflection member may be changed to a small sized magnet. Further, it is configured that, when the reflection member 109 reaches immediately beneath of the reflection type optical sensor 108, the reflection type optical sensor 108 outputs an H level signal. However, the sensor may be configured to output an L level signal when the reflection member 109 reaches immediately beneath of the reflection type optical sensor 108.

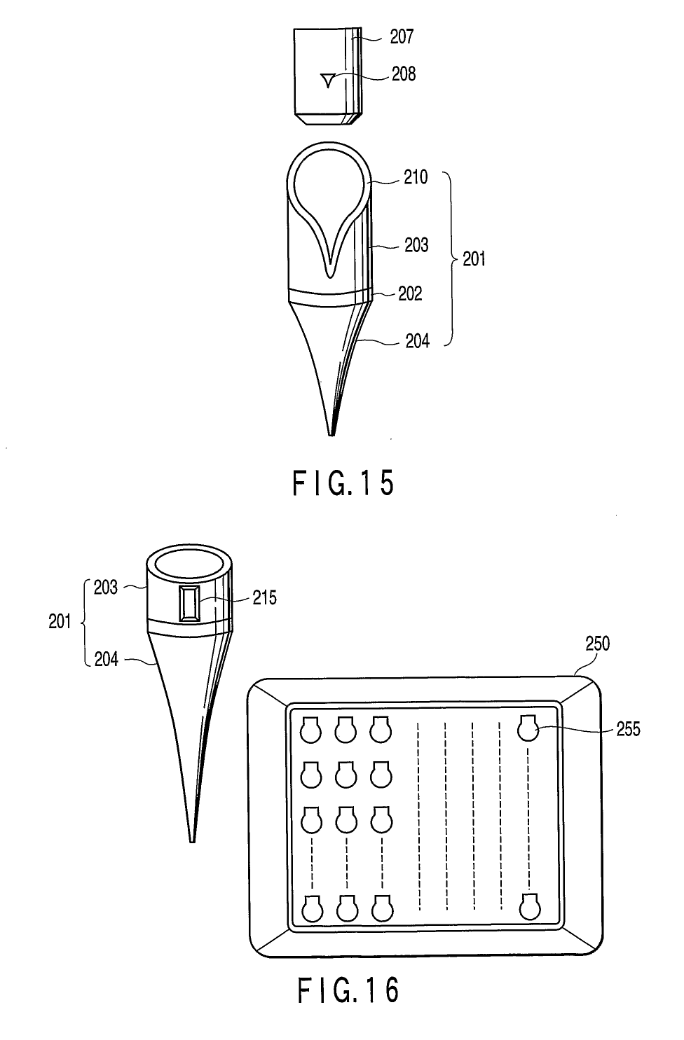

In addition, the reaction container may be formed in the shape shown in FIG. 15. As shown in FIG. 15, the guide cutout section 210 of the reaction container 201 is formed in a shape which is different from that shown in FIG. 13, so that the engagement section 207 and the coupling section 203 engage with each other in an always constant direction. By forming such a shape, for example, a reflection type optical sensor or a reflection member for determining an engaging direction can be eliminated.

Further, as shown in FIG. 16, a protrusion section 215 is provided at the coupling section 203 of the reaction container 201 and is formed in a shape such that the protrusion section 215 engages with a hole 255 for setting the reaction container 201 of a plate 250 on which the reaction container 201 has been set. In this manner, at a time point at which the reaction container 201 has been set on the plate 250, the reaction container 201 is oriented in a fixed direction. Therefore, at a time point at which the engagement section 207 and the coupling section 203 engage with each other, the reaction container 201 is oriented in a fixed direction, and thus, there is no need for any special mechanism.

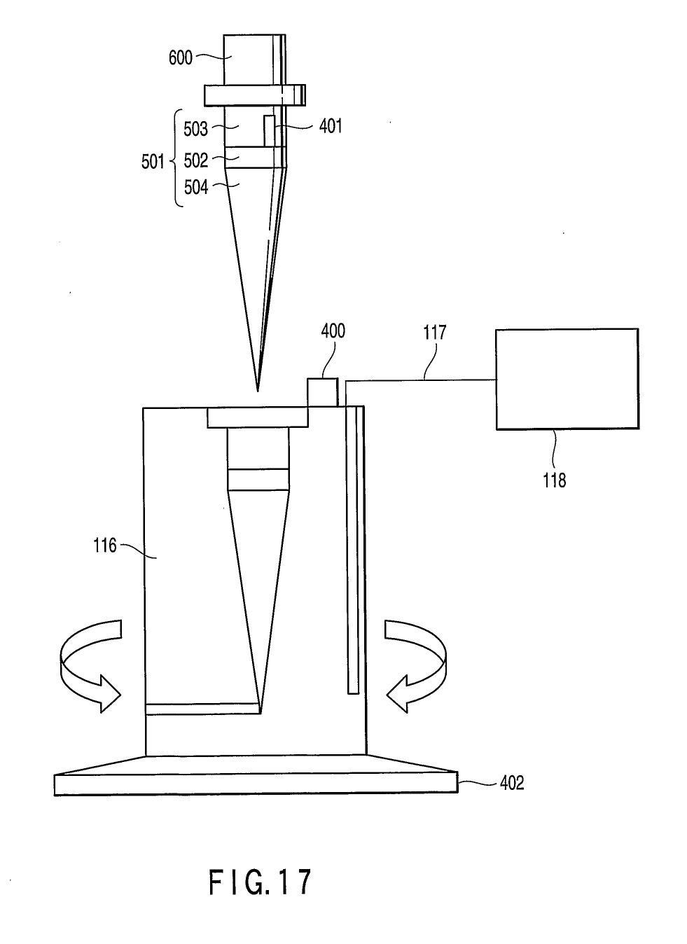

According to still another modification, a configuration shown in FIG. 17 is possible. In FIG. 17, the heater 116 is mounted on an automatic rotary base 402 so as to be rotatable by the automatic rotary base 402. In addition, an optical sensor 400 is provided at the heater 116. A reflection member 401 is mounted on a side face of a reaction container 501, and the reaction container 401 is detected by the optical sensor 400, as described later.

In the above-described configuration, the reaction container 501 is generally mounted so as to come into intimate contact with a recess section of the heater 116. In this modification, however, the reaction container 501 stops temporarily immediately before the container comes into intimate contact with the recess section of the heater 116. Then, while a position of the reaction container 501 is maintained, the heater 116 starts its rotation by means of the automatic rotary base 402. When the reflection member 401 is detected by the optical sensor 400, the rotation of the heater 116 is stopped, the position information (that is, rotary angle information on the heater 116) is transmitted to a CCD camera rotation mechanism (not shown), and the CCD camera is rotated on the basis of the angle information. Thereafter, the reaction container 501 is lowered and is brought into intimate contact with the heater 116 to carry out hybridization. After hybridization has finished, an electrically driven slider (not shown) is rotated, the reaction container 501 is removed from an engagement section 600, and the light from a probe spot on a three-dimensional substrate 502 is picked up by the CCD camera. Consequently, since the orientations of the CCD camera and the reaction container 501 match each other, precise data analysis can be carried out on the basis of a positional relationship of the photographed spot. In this modification, rotation of the CCD camera may not be carried out before hybridization or may be carried out during hybridization or before photographing which follows hybridization. In addition, the picked-up image may be rotated by image processing on the basis of a detected angle without rotating the CCD camera.

Moreover, the orientation on the picked-up image has been fixed by the orientation of the CCD camera or image processing because the position of the reaction container 501 is not set in a fixed direction in the above embodiment, but the following procedure may be taken. First, as described above, the reaction container 501 is lowered to come into intimate contact with the heater 116 to carry out hybridization after the heater 116 has been rotated on the basis of angle information. After hybridization has finished, an electrically driven slider (not shown) is rotated, the reaction container 501 is removed from the engagement section 600, and the position of the heater 115 is reset to an initial position (that is, a position before rotated). In this manner, the reaction container 501 is always oriented in a fixed direction.

Further, inventions of a variety of stages are included in each of the above embodiments, and a variety of the inventions can be excerpted in accordance with a proper combination in a plurality of disclosed constituent elements.

For example, even if some of the constituent elements are deleted from all of the constituent elements shown in the embodiments, a configuration from which the above constituent elements have been deleted can be excerpted as a statuary invention in the case where the problems described in the BACKGROUND ART section can be solved and the advantageous effect described in the Advantage Effect of the Invention can be attained.

According to the present invention, since the direction of a reaction container (that is, a substrate) is always fixed with respect to the image pickup direction of detection means such as a CCD camera, the image pickup direction of the detection means such as a CCD camera and the spot array direction can be coincided with each other, thus making it possible to carry out precise data analysis based on a positional relationship of the photographed spot. Further, even in the case where the image pickup direction and the array direction do not coincide with each other, a deviation of a rotary angle of a picked-up image is corrected, so that it is possible to carry out precise data analysis based on a spot positional relationship. Additionally, the present invention is applied to a work of assembling a three-directional substrate in a reaction container, thereby making it possible to automatically and efficiently carry out a process for aligning the directions of the reflection member and the three-dimensional substrate embedded in a coupling section, for example.

### INDUSTRIAL APPLICABILITY

The present invention relates to a reaction container provided to detect a biologically associated substance, and a reaction device and a detection device using the reaction container.

1. A reaction container having a substrate in which a probe to detect a biologically associate substance is phase-locked, the reaction container comprising a correcting section to align a detection direction of a detector to detect a reaction of the substrate and a direction of the substrate.

2. A reaction container according to claim 1, wherein the correcting section is any of a directional detecting member provided in the reaction container, a cutout for directional correction, and a protrusion for directional correction.

3. A reaction container according to claim 1, wherein the substrate is a three-dimensional substrate.

4. A reaction container according to claim 1, further comprising:
a solution charge section configured to charge a solution; and
a coupling section configured to couple the container with liquid drive means driven in the reaction container with air tightness and to couple the container with the detector so as to prevent leakage of light, wherein
the substrate is allocated between the coupling section and the solution charge section such that directions of the substrate and the coupling section match each other.

5. A detection device comprising:
a reaction container having a substrate in which a probe to detect a biologically associated substance is phase-locked;
a detector which detects a reaction of the substrate; and
a correcting section to match a direction of the substrate and a detection direction of the detector.

6. A detection device according to claim 5, wherein the correcting section rotates the reaction container in order to detect a directional marker provided at a predetermined position of the reaction container and corrects a direction of the reaction container by detecting the reaction container.

7. A detection device according to claim 5, wherein the correcting section detects a direction of the reaction container and carries out image processing for an image obtained by a detection result of the detector on the basis of the detected direction of the reaction container, thereby correcting a direction of the substrate.

8. A detection device according to claim 5, wherein the correcting section is a cutout provided in the reaction container and corrects a direction when engaging with an engagement section to be engaged with the reaction container so as to be oriented in a predetermined direction.

9. A reaction device comprising:
a reaction container having a substrate in which a probe to detect a biologically associated substance is phase-locked;
a pressure transmitting section which transmits to the reaction container a pressure for charging and discharging a sample solution;
a temperature adjusting section which adjusts a temperature of the sample solution contained in the reaction container; and
a correcting section which causes the reaction container to be oriented in a predetermined direction.
